(19) United States
(12) Patent Application Publication
(10) Pub. No.: US 2025/0367000 A1
(43) Pub. Date: Dec. 4, 2025

(54) Glenoid Baseplate and Manufacturing Method Thereof

(71) Applicant: CORENTEC CO., LTD, Seoul (KR)

(72) Inventors: Jung-Sung Kim, Seoul (KR); Jae-Won Kim, Seoul (KR); Sung-Wook Jung, Seoul (KR); Sang-Kil Lee, Hanam-si, Gyeonggi-do (KR); Yeon-Beom Heo, Seoul (KR)

(21) Appl. No.: 18/706,153

(22) PCT Filed: Oct. 17, 2022
(86) PCT No.: PCT/KR2022/015686

§ 371 Date: Jan. 28, 2025

(30) Foreign Application Priority Data
Nov. 3, 2021 (KR) 10-2021-0149534

(51) Int. Cl. A61F 2/40; A61F 2/30

(52) U.S. Cl. CPC A61F 2/4081; A61F 2/30749; A61F 2002/30011; A61F 2002/30403; A61F 2002/30785; A61F 2002/30879; A61F 2002/30901; A61F 2002/30985

(57) ABSTRACT

A glenoid baseplate, for an artificial shoulder joint, includes: a base having a fixing hole formed so as to insertedly accommodate a fixer; an insertion part extending at a predetermined angle from the base; and a recessed part made by forming a recess in a part of one surface of the base which is located a predetermined distance from at least a central fixing hole, a peripheral fixing hole, and the edge of the base. The base includes: the central fixing hole formed vertically through the base; and the peripheral fixing hole formed in the periphery of the central fixing hole. The insertion part includes a shaft extending from the base so as to have a hollow extending from the central fixing hole, the insertion part including: a rib protruding, so as to have a predetermined width, from one base-side end of the shaft to the other end; and a rim protruding from the shaft.

### TECHNICAL FIELD

The present disclosure relates to a glenoid baseplate and a manufacturing method thereof. More particularly, the present disclosure relates to a glenoid baseplate of an artificial shoulder joint and a manufacturing method of the glenoid baseplate. The glenoid baseplate includes a base configured to be settled on a scapular glenoid and having a fixing hole formed to penetrate and accommodate a fixing means, and an insertion part extending at a predetermined angle from one side of the base. The base includes a central fixing hole formed vertically through the base, and a peripheral fixing hole formed around the central fixing hole. The insertion part includes a shaft extending from one surface of the base having a hollow that extends from the central fixing hole, a rib with a predetermined width and extending from one end of the base to the other end of the base of the shaft, and a rim protruding in a ring shape with a predetermined width from at least one end of the shaft. The glenoid baseplate further includes a recessed part recessed a portion of at least one surface of the base spaced a predetermined distance apart from at least an edge of the central fixing hole, the peripheral fixing hole, and the base. Furthermore, the present disclosure minimizes a solid region at an appropriate strength by applying a phase optimization design technology, induces maximum bone ingrowth by maximizing a porous structure region, and reduces manufacturing cost and time by laminating even a porous surface all at once by applying a 3D printing manufacturing method.

### BACKGROUND ART

An artificial shoulder joint is a type of artificial prosthesis that replaces the shoulder joint of a human when the shoulder joint is not functioning properly. The artificial shoulder joint includes a stem implanted into the humerus, a glenoid baseplate coupled to the shoulder blade (scapula), and an artificial bone head and an insert that implements a rotational movement between the stem and the glenoid baseplate. The artificial bone head may be coupled to a stem like the shoulder joint of a human, or vice versa to the glenoid baseplate. Accordingly, the insert is respectively coupled to the glenoid baseplate and the stem.

Referring to FIG. 1, scapula 91 has a substantially inverted triangle structure, and includes subscapular fossa 913 that is a front surface of a body facing an anterior direction, infraspinous fossa (not illustrated) that is a rear surface of the body facing a posterior direction, coracoid 917 that protrudes from an upper side toward the anterior direction, acromion 919 that protrudes from an upper side toward the posterior direction, and glenoid 911 which faces a lateral direction from between the coracoid 917 and the acromion and which is in contact with the caput humeri to implement a joint movement.

Generally, when a total joint arthroplasty of an artificial shoulder joint is performed, a glenoid baseplate is coupled to the glenoid 911 so that the glenoid baseplate replaces a function of the glenoid 911. At this time, a fixing means such as a screw is used to securely couple the glenoid baseplate to the glenoid 911.

### Patent Document

U.S. Patent No. U.S. Pat. No. 9,132,016 (registered on Sep. 15, 2015) “Implantable shoulder prostheses”

The invention disclosed in the patent document described above discloses a glenoid baseplate used in an artificial shoulder joint.

However, the prior art is generally manufactured in the same manner as in FIG. 2. When the glenoid baseplate is manufactured through conventional machining, a large amount of material loss occurs, and a complicated procedure is required to implement a porous surface. In addition, when a predetermined thickness is secured to express a constant strength, bone growth is limited because it is impossible to implement a complicated shape through machining.

### DISCLOSURE

### Technical Problem

Accordingly, the present disclosure has been made keeping in mind the above problems occurring in the related art.

An objective of the present disclosure is to provide a glenoid baseplate capable of being stably coupled to the glenoid, the glenoid baseplate including a base which has a fixing hole formed to insertedly accommodate a fixing means and mounted on the scapular glenoid and an insertion part extending with a predetermined angle. The base includes a central fixing hole formed vertically through the base and including a peripheral fixing hole formed around the central fixing hole, and the insertion part includes a shaft extending from one surface of the base to have a hollow extending from the central fixing hole.

Another objective of the present disclosure is to provide a glenoid baseplate capable of reducing the weight of the insertion part while providing enough strength to withstand athletic loads on the shoulder joint, wherein the insertion part comprises a reinforcement member extending along outer surface, and the reinforcement member comprises a rib extending with a predetermined width from one end of the base of the stem to the other end, and a rim protruding in a ring shape from at least one end of the shaft.

In addition, still another objective of the present disclosure is to provide a glenoid baseplate optimizing the expression of strength for the volume of the reinforcement member, wherein the rib is formed to gradually decrease in width as the rib protrudes from outer surface of the shaft.

In addition, yet another objective of the present disclosure is to provide a glenoid baseplate including a recessed part recessed by a predetermined depth in at least one surface of a base, thereby making the base lightweight.

In addition, yet another objective of the present disclosure is to provide a glenoid baseplate having different thickness between the relatively strongly stressed portion and weakly stressed portion to express appropriate strength and maximize weight lightening by forming a recessed part formed in at least one surface of the base is recessed at least a predetermined distance from the edge of at least one of the central fixing hole, the peripheral fixing hole, and the edge of the base.

In addition, yet another objective of the present disclosure is to provide a glenoid baseplate that prevents the porous layer from being broken by further including a flange that protrudes from one surface of a base along an edge of a peripheral fixing hole, thereby being capable of preventing damage to a porous layer.

In addition, yet another objective of the present disclosure is to provide a glenoid baseplate capable of promoting bone growth between pores by comprising a porous layer formed to coat surface of the base and the stem with a predetermined thickness on a side of the base and the stem, with plural pores therein, and having a shape complementary to the base and the stem.

In addition, yet another objective of the present disclosure is to provide a manufacturing method of a glenoid baseplate capable of minimizing material consumption, wherein the manufacturing method includes a shape determination step determining a shape of the glenoid baseplate, an optimization step deriving an optimal shape of the glenoid baseplate on the basis of a load and a restriction condition acting on the glenoid baseplate, a detailed design step determining a detailed shape of the glenoid baseplate according to the optimal shape determined in the optimization step, and an additive manufacturing step manufacturing a determined solid region and a determined porous layer in a stacking manner.

In addition, yet another objective of the present disclosure is to provide a manufacturing method of a glenoid baseplate capable of designing a shape of the glenoid baseplate with maximized bone growth and weight lightening, wherein the manufacturing method includes an optimization step and a detailed design step. The optimization step includes a region setting step setting a region to be optimized through analysis, a restriction condition setting step setting an optimization restriction condition together with an objective function that is an objective of optimization, a load determination step setting a load and a constraint condition applied to the glenoid baseplate, and a calculation step deriving the optimal shape. The detailed design step includes a reinforcement member forming step determining a reinforcement member formed around an insertion part of the glenoid baseplate, a recessed part forming step determining a recessed part recessed from a base of the glenoid baseplate, and a porous layer forming step determining pores and thickness of the porous layer formed to coat a surface of the solid region formed by the base and the insertion part.

In addition, yet another objective of the present disclosure is to provide a manufacturing method of a glenoid baseplate, the manufacturing method being capable of reducing the manufacturing cost and time since a solid region and a porous layer are stacked with the same material.

### Technical Solution

The present disclosure may be implemented by one or more embodiments having some or all of the following configurations, to achieve one or more of the above-described objectives.

According to an embodiment of the present disclosure, the present disclosure comprises a base having a fixing hole formed to insertedly accommodate a fixing means and mounted on the scapular glenoid; and an insertion part having a predetermined angle and extending from one side of the base, wherein the base includes a central fixing hole formed vertically through the base and a peripheral fixing hole formed around the central fixing hole, and the insertion part includes a shaft extending from one surface of the base to form a hollow that extends from the central fixing hole.

According to another embodiment of the present disclosure, the insertion part of the present disclosure may include a reinforcement member protruding along an outer circumferential surface of the insertion part, and the reinforcement member may include a rib with a predetermined width protruding and extending from a one end of the base to the other end of the base of the shaft.

According to still another embodiment of the present disclosure, the reinforcement member of the present disclosure may further include a rim protruding in a ring shape from at least one end of the shaft with a predetermined width.

According to yet another embodiment of the present disclosure, the width of the rib is gradually decreased as the rib protrudes from an outer surface of the shaft.

According to yet another embodiment of the present disclosure, the glenoid baseplate may include a recessed part recessed by a predetermined depth in at least one surface of the base.

According to yet another embodiment of the present disclosure, the recessed part of the present disclosure may be formed by recessing a portion in one surface of the base, the portion being spaced a predetermined distance apart from at least one of an edge of the central fixing hole, the peripheral fixing hole, and the base.

According to yet another embodiment of the present disclosure, the base of the present disclosure may further include a flange protruding from one surface of the base along the edge of the peripheral fixing hole.

According to yet another embodiment of the present disclosure, the glenoid baseplate may include a porous layer having a predetermined thickness on one side of the base and the stem, which is formed to coat the surface of the base and the stem and has plural pores therein, wherein the porous layer has a complementary shape to the base and the stem so that bone growth can be promoted between pores.

According to yet another embodiment of the present disclosure, an extended end of the shaft and an edge of the peripheral fixing hole may be exposed and may not be covered by the porous layer.

According to yet another embodiment of the present disclosure, the present disclosure comprises a manufacturing method of a glenoid baseplate, the manufacturing method including: a shape determination step determining a shape of the glenoid baseplate; an optimization step deriving an optimal shape of the glenoid baseplate on the basis of a load and a restriction condition acting on the glenoid baseplate; a detailed design step determining a detailed shape of the glenoid baseplate according to the optimal shape determined in the optimization step; and an additive manufacturing step manufacturing a determined solid region and a determined porous layer in a stacking manner.

According to yet another embodiment of the present disclosure, the optimization step of the present disclosure may include: a region setting step setting a region to be optimized through analysis; a restriction condition setting step setting an optimization restriction condition together with an objective function that is an objective of optimization; a load determination step setting a load and a constraint condition applied to the glenoid baseplate; and a calculation step deriving the optimal shape.

According to yet another embodiment of the present disclosure, the detailed design step of the present disclosure may include: a reinforcement member forming step determining a reinforcement member formed around an insertion part of the glenoid baseplate; a recessed part forming step determining a recessed part formed by being recessed from a base of the glenoid baseplate; and a porous layer forming step determining a porosity and a thickness of the porous layer that is formed coat a surface of the solid region formed of the base and the insertion part.

According to yet another embodiment of the present disclosure, the solid region and the porous layer may be stacked with the same material.

### Advantageous Effects

The present disclosure may achieve the follow effects from the embodiment and configurations described below, as well as combinations and relationships of use thereof.

According to the present disclosure, the glenoid baseplate includes the base which has the fixing hole formed to insertedly accommodate the fixing means and which is mounted on the scapular glenoid and includes the insertion part which has the predetermined angle and which extends from one side of the base. The base includes the central fixing hole formed vertically through the base and the peripheral fixing hole formed around the central fixing hole, and the insertion part includes the shaft that extends from one surface of the base such that the base has a hollow that extends from the central fixing hole. Therefore, there is an effect that the glenoid baseplate capable of being stably coupled to the glenoid is provided.

According to the present disclosure, the insertion part includes the reinforcement member protruding along the outer circumferential surface of the insertion part, and the reinforcement member includes a rib with a predetermined width protruding and extending from the one end of the base to the other end of the base of the shaft and a rim in a ring shape protruding from the shaft from at least one end of the shaft with a predetermined width. Therefore, it is possible to provide a glenoid baseplate capable of exhibiting strength that can withstand the load according to the movement of the shoulder joint while the insertion part is made lightweight.

According to the present disclosure, the rib is formed to gradually decrease in width as the rib protrudes from the outer surface of the shaft, thereby the strength exhibition compared to the volume of the reinforcement member may be optimized.

According to the present disclosure, the present disclosure comprises a recessed part recessed in at least one surface of the base in a predetermined depth, thereby making the base lightweight.

According to the present disclosure, a recessed part recessed at a predetermined depth in at least one surface of the base is recessed at a predetermined distance from the edge of at least one of the central fixing hole, the peripheral fixing hole, and the edge of the base, so that the thickness of the relatively strongly stressed portion and weakly stressed portion are made differently, thereby expressing appropriate strength and maximizing weight lightening.

According to the present disclosure, the base further includes a flange protruding from one surface of the base along the edge of a peripheral fixing hole, thereby preventing damage to the porous layer.

According to the present disclosure, the present disclosure includes a porous layer with predetermined thickness and an inner portion having plural pores formed to coat surfaces of the base and the insertion part, and the porous layer has a complementary shape to the base and the insertion part, thereby maximizing bone growth.

According to the present disclosure, the manufacturing method of the glenoid baseplate includes the shape determination step determining the shape of the glenoid baseplate, the optimization step deriving the optimal shape of the glenoid baseplate on the basis of the load and the restriction condition acting on the glenoid baseplate, the detailed design step determining the detailed shape of the glenoid baseplate according to the optimal shape determined in the optimization step, and the additive manufacturing step manufacturing the determined solid region and the determined porous layer in the stacking manner, thereby minimizing material consumption.

According to the present disclosure, the optimization step includes the region setting step setting the region to be optimized through analysis, the restriction condition setting step setting the optimization restriction condition together with the objective function that is the objective of optimization, the load determination step setting the load and the constraint condition applied to the glenoid baseplate, and the calculation step deriving the optimal shape. The detailed design step includes the reinforcement member forming step determining the reinforcement member formed around the insertion part of the glenoid baseplate, the recessed part forming step determining the recessed part formed by being recessed from the base of the glenoid baseplate, and the porous layer forming step determining the porosity and the thickness of the porous layer that is formed so as to coat the surface of the solid region formed of the base and the insertion part, thereby providing the shape of the glenoid baseplate maximizing the bone growth and making the glenoid baseplate lightweight.

According to the present disclosure, the solid region and the porous layer are stacked with the same material, thereby providing a manufacturing method of glenoid baseplate reducing manufacturing cost and time.

### DESCRIPTION OF DRAWINGS

FIG. 1 is a perspective view illustrating a glenoid baseplate and a scapula according to a conventional technology.

FIG. 2 is a view illustrating manufacturing step of the glenoid baseplate according to the prior art by machining.

FIG. 3 is a perspective view illustrating a glenoid baseplate according to an embodiment of the present disclosure.

FIG. 4 is an exploded perspective view illustrating the glenoid baseplate according to an embodiment of the present disclosure.

FIG. 5 is a plan view illustrating the glenoid baseplate according to an embodiment of the present disclosure when viewed from one side.

FIG. 6 is a cross-sectional view of the glenoid baseplate according to an embodiment of the present disclosure taken along line A-A′.

FIG. 7 to FIG. 10 are views illustrating a state in which a rib 135a and a second rib 135c are formed on a shaft 131 according to various embodiments of the present disclosure.

FIG. 11 is a perspective view illustrating a porous layer 30 according to an embodiment of the present disclosure.

FIG. 12 is an exploded perspective view illustrating the glenoid baseplate according to another embodiment of the present disclosure.

FIG. 13 is a plan view illustrating the glenoid baseplate according to another embodiment of the present disclosure.

FIG. 14 is a view illustrating a fixing means 8 according to an embodiment of the present disclosure.

FIG. 15 is a flowchart illustrating a manufacturing method of a glenoid baseplate according to an embodiment of the present disclosure.

FIG. 16 is a flowchart illustrating an optimization step S20 according to an embodiment of the present disclosure.

FIG. 17 is a view illustrating a load acting on the scapula according to a movement of the shoulder joint.

FIG. 18 is a conceptual view illustrating an additive manufacturing step S50 according to an embodiment of the present disclosure.

### MODE FOR INVENTION

Hereinafter, a glenoid baseplate of an artificial shoulder joint according to the present disclosure will be described in detail with reference to the accompanying drawings. It should be noted that like elements are indicated by like reference numerals throughout the drawings wherever possible. In addition, a detailed description of known functions and configurations incorporated herein may be omitted when it may obscure the subject matter of the present disclosure. Unless there is a special definition, all terms in this specification are the same as the general meaning of terms understood by those skilled in the art to which the present disclosure belongs, and when conflicting with the meaning of terms used in this specification, the terms used generally in the art follow the definition of the terms used herein. Throughout the specification, it will be understood that when a part is referred to as “including” an element, the part does not preclude other elements and may further include other elements unless stated otherwise. In addition, terms such as “part” and so on refer to units which perform at least one function or operation. In addition, when components are referred to as “connected”, it may mean that the components are not limited to being engaged in direct contact with each other, but includes being engaged through another component, and may be disposed such that a predetermined force or energy is capable of being transmitted even if the component is not engaged. Terms such as “first” and “second” may be used to indicate the same or substantially the same configuration in a different order, and may be interpreted as substantially the same configuration as a configuration that does not indicate “first”, “second”, and so on. Hereinafter, an exemplary embodiment of the present disclosure will be described with reference to the accompanying drawings to describe the present disclosure in detail.

Referring to FIG. 3, a glenoid baseplate 1 according to the present disclosure forms an artificial shoulder joint together with other surgical apparatus such as a glenosphere, an insert, and so on. As described later, the glenoid baseplate 1 accommodates a fixing means 8, and the fixing means is engaged with the bone or is inserted into the bone, so that the glenoid baseplate 1 is capable of being fixed to glenoid 911. The glenoid baseplate 1 may include a solid region 10 and a porous layer 30.

The solid region 10 is a portion exhibiting a strength of the baseplate. In an embodiment, the solid region 10 may be provided such that the solid region 10 exhibits a required strength while being made lightweight. Here, a solid is a concept that contrasts with a porosity. That is, the solid may be understood as a shape that is smooth, hard, or has no pores, unlike the porous layer 30 in which a plurality of pores is formed. Each configuration of the solid region 10 has the same overall size compared to a conventionally used baseplate, but a portion that has little influence on exhibiting the strength is omitted for realizing bone growth and making the glenoid baseplate lightweight, so that each configuration of the solid region 10 may have a width or a thickness smaller than a width or a thickness of a conventional baseplate. The solid region 10 may include a base 11 mounted on the glenoid 911, and may include an insertion part 13 that extends at a predetermined angle from one side of the base 11.

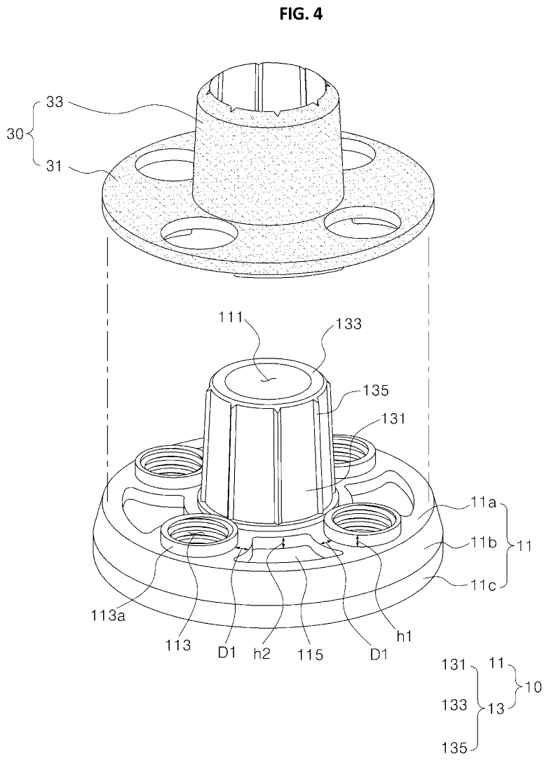

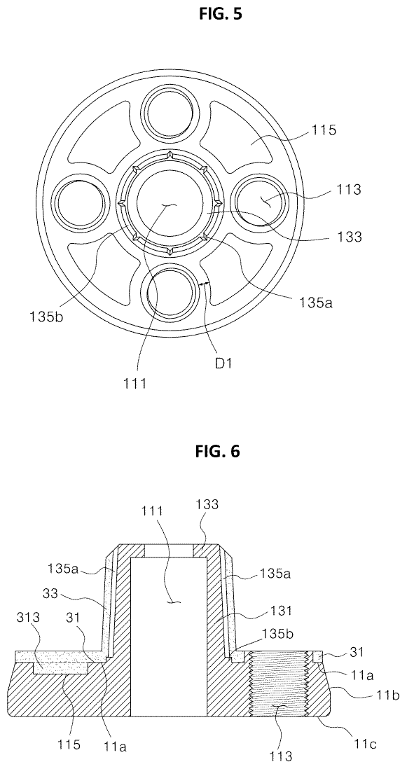

Referring to FIG. 4 and FIG. 5, the base 11 may be formed in a plate shape having a predetermined thickness. In an exemplary embodiment, the base 11 may have a circular plate shape. However, in another embodiment to be described later, the base may have an atypical shape or an asymmetrical shape that is not a circular shape. The base 11 includes an upper surface 11a and a lower surface 11c, and is surrounded by a side surface 11b connecting the upper surface 11a and the lower surface 11c to each other. Therefore, the base 11 may be a solid object disposed between the upper surface 11a and the lower surface 11c. Although the upper surface 11a and the lower surface 11c may be formed such that the upper surface 11a and the lower surface 11c are substantially flat or not having curvatures, the upper surface 11a and the lower surface 11c may be curved such that the upper surface 11a and the lower surface 11c have predetermined curvatures. Particularly, the upper surface 11a in a direction in contact with the bone may have a convex shape suitable for being fixed to the glenoid 911. In addition, the base 11 includes a central fixing hole 111 that passes through the upper surface 11a and the lower surface 11c and a peripheral fixing hole 113 as fixing holes. Furthermore, the base 11 may further include a recessed part 115 formed by being recessed to a predetermined depth in at least one of a surface of the base 11.

The central fixing hole 111 is formed vertically through the base. The central fixing hole 111 is formed in the center of the base 11, and the fixing means to be described later is inserted into and is passing through the central fixing hole 111. Although the central fixing hole 111 is capable of being disposed on an axis that vertically passes through the base 11 and/or the glenoid baseplate 1, the center of the central fixing hole 111 is not required to be aligned with the axis. Furthermore, even when the central fixing hole 111 is formed on a position that is deviated from the center, it can be understood as a concept that the central fixing hole 111 extends in a direction in which the upper surface 11a and/or the insertion part 13 that will be described later are formed in contrast to the peripheral fixing hole 113.

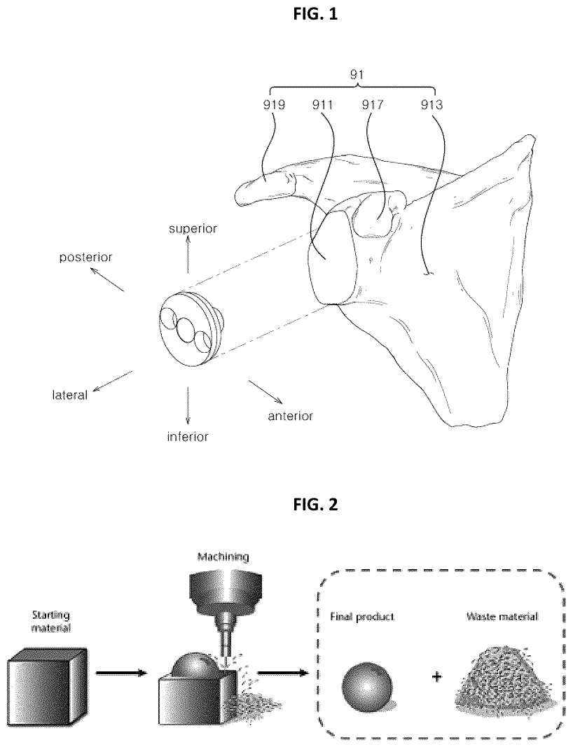

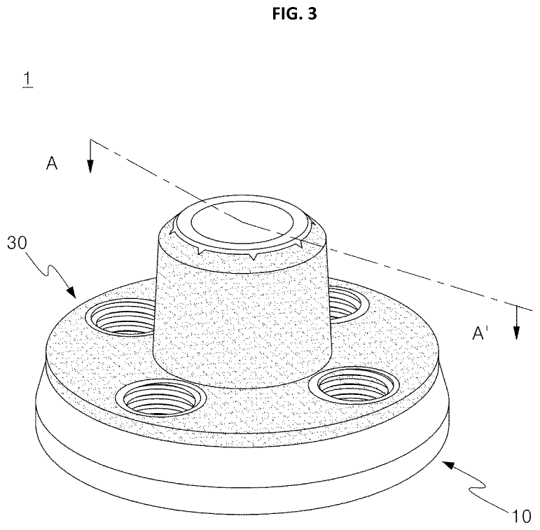

The peripheral fixing hole 113 is formed around the central fixing hole 111. In an embodiment of the present disclosure, a total of four peripheral fixing holes 113 may be formed, one in each of four directions of the central fixing hole 111. However, this is only an embodiment of the present disclosure, and there is no limitation in the number and direction of the peripheral fixing holes 113 and/or a central angle between the centers of the peripheral fixing hole 113 with the central fixing hole 111 as the center. The peripheral fixing hole 113 may be defined as a hole defined by an inner circumferential surface that extends from the upper surface 11a to the lower surface 11c, and may be formed in a tapered shape in which a width thereof becomes gradually reduced downward. In addition, the peripheral fixing hole 113 may be formed by forming a predetermined angle that is not perpendicular to the glenoid 911 (see FIG. 1). This means that an opening of the peripheral fixing hole that is formed through the upper surface 11a and the lower surface 11c may be an eccentric circle. In particular, when the peripheral fixing hole 113 is formed from the upper surface to the lower surface, the peripheral fixing hole 113 may be formed in a direction that is inclined outward. Here, the outward refers to a direction away from the center of the central fixing hole 111. A thread is formed on the inner circumferential surface of the peripheral fixing hole 113, and the fixing means having a corresponding thread may be fixed to the peripheral fixing hole 113.

The base 11 may include a flange 113a that protrudes from one surface of the base 11 along an edge of the peripheral fixing hole 113. The flange 113a may be formed for exhibiting a proper strength of the solid region 10. A larger stress may be generated around the central fixing hole 111 and/or the peripheral fixing hole 113 by the fixing means than in other portions of the solid region 10, and the flange 113a may be provided such that the flange 113a forms the strength against such stress. Furthermore, the flange 113a may be provided such that the flange 113a is exposed toward a bone contact surface when the porous layer 30 that will be described later is coated and/or formed on the solid region 10. When a fixing member is inserted into a bone region through the peripheral fixing hole 113, the flange 113a may guide the fixing member. The flange 113a may protrude from one surface of the base by a height of h1.

Referring to FIG. 4 to FIG. 6, the recessed part 115 is formed at the predetermined depth in one surface of the base 11, and it is preferable that the recessed part 115 is formed in a direction in contact with the glenoid. That is, it is preferable that the recessed part 115 is formed in the upper surface 11a on which the porous layer 30 is formed. The recessed part 115 may be formed by recessing a portion that is spaced apart by a predetermined distance from at least one of the central fixing hole, the peripheral fixing hole, and an edge of the base. In an exemplary embodiment, as illustrated in FIG. 5, the recessed part 115 may be formed in a portion spaced apart by a length of D1 from the peripheral fixing hole 113 and is recessed by a height of h2 while forming a boundary with the upper surface 11a. By recessing a portion where a relatively small stress is applied, a space in which the bone can grow that will be described later may be maximized and sufficient strength may be exhibited. As such, the recessed part 115 may be formed such that the recessed part 115 is spaced apart by a predetermined distance from the central fixing hole 111 and the edge of the base 11. The distance at which the boundary of the recessed part 115 is spaced apart from the peripheral fixing hole 113, the central fixing hole 111, and the edge of the base may vary according to a shape of the baseplate and the number of fixing holes. In another embodiment, the recessed part 115 may be formed in the lower surface 11c or may be formed at a predetermined depth in both the upper surface 11a and the lower surface 11c, thereby being capable of minimizing a thickness of a portion of the base 11 having a small influence on the strength exhibition of the baseplate. Several recessed parts 115 may be formed according to a shape of the base 11. Furthermore, the several recessed parts 115 may be recessed by a different depth for each portion, or a recessed depth in one recessed part 115 may be gradually changed.

Referring to FIG. 4 and FIG. 6, the insertion part 13 is a configuration that extends toward a direction from the upper surface 11a of the base 11. Preferably, the insertion part 13 may have a cylindrical column shape, and may be inserted into the glenoid 911 of the scapula 91. At this time, in order to secure an optimal fixing force between the scapula 91 and the glenoid baseplate 1, it is preferable that an axis of the insertion part 13 is vertically deployed to the glenoid 911. The insertion part 13 has an inner portion formed in a hollow shape, and may extend from the central fixing hole. The insertion part 13 may include a shaft 131 that extends and forms a hollow, a support 133 deployed radially inward from one end of the shaft 131 by a predetermined length, and a reinforcement member 135 that protrudes radially outward from the shaft 131.

The shaft 131 forms the hollow which is in communication with the central fixing hole 111 and which is a passage where the fixing means passes therethrough. The shaft 131 may have a cylindrical column shape having a predetermined inner diameter. In some embodiments, the shaft 131 may be formed such that the shaft 133 is tapered as the shaft 131 extends from the base to an end of an upper side of the base. As described above, the shaft 131 may be formed on the base 11 such that the shaft has a thickness smaller than a thickness of a conventional baseplate.

The support 133 is a configuration in which the fixing means that passes through the central fixing hole 111 is caught so that a position is specified, and a center of the support 133 is perforated such that the fixing means is capable of being inserted into the glenoid. In addition, an outer edge of the support 133 is chamfered, so that the support 133 is capable of being easily inserted into the glenoid 911 of the scapula 91.

The reinforcement member 135 is a configuration that protrudes along an outer circumferential surface of the shaft 131 and/or the insertion part 13, and may be provided such that the rigidity and/or the strength of the shaft 131 is reinforced. The reinforcement member 135 may be formed of a plurality of rims having a predetermined width, and will be described by dividing the reinforcement member 135 into a rib 135a and a rim 135b.

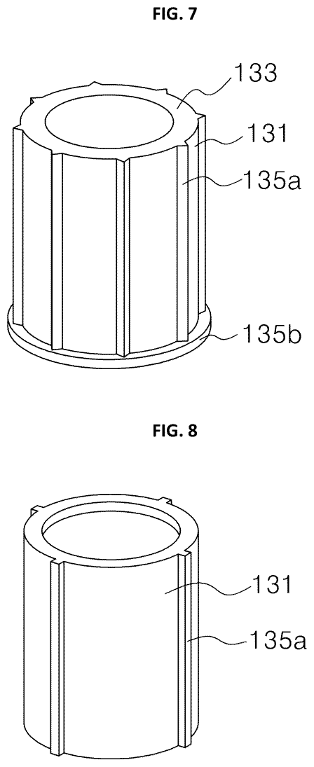

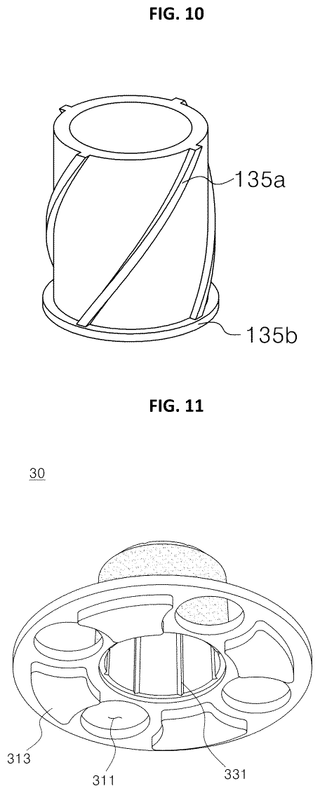

As illustrated in FIG. 7 to FIG. 10, the rib 135a and the rim 135b protrude along the outer circumferential surface of the shaft 131 by a predetermined width and/or a predetermined thickness. The rib 135a protrudes and extends in a longitudinal direction of the shaft by the predetermined width. The rib 135a may extend from one end of the base to the other end of the base of the shaft. Accordingly, the rib 135a may extend vertically at a predetermined center angle from a center of the shaft 131. In an exemplary embodiment of the present disclosure, eight ribs 135a may be formed at a center angle of 45 degrees from the center of the shaft. In another embodiment, four, twelve, or ten ribs 135a may be formed, and the center angle of the plurality of ribs 135a from the center of the shaft 131 may be not constant and may be different from each other. Particularly, the rib 135a may be formed such that the rib 135a is concentrated on a predetermined portion of the shaft according to a movement shape of the shoulder joint and/or the shape of the baseplate, which will be described later. In addition, as illustrated in FIG. 10, the rib 135a may not extend straight, but may extend on the shaft 131 such that the rib 135a has a spiral shape.

The rib 135a may be formed such that the width of the rib 135a gradually decreases as the rib 135a protrudes from an outer surface of the shaft. Accordingly, a cross-sectional shape of the rib 135a may have a shape that is tapered toward the outside, and may preferably have a triangular cross-sectional shape. In another embodiment, a cross-section of the rib 135a may have a trapezoidal shape in which the width is gradually decreased toward the outside, or may have a substantially rectangular shape in which the width is not gradually decreased. The same may be applied to the rim 135b that will be described later.

The rim 135b has a ring shape having a predetermined width at least one end of the shaft and protrudes from the shaft. The rim 135b may be formed in an annular shape from one end of the base of the shaft 131, or may be formed in an annular shape from one end in a direction in which the rim 135b is inserted into the glenoid, or may be formed in an annular shape from both ends of the shaft 131.

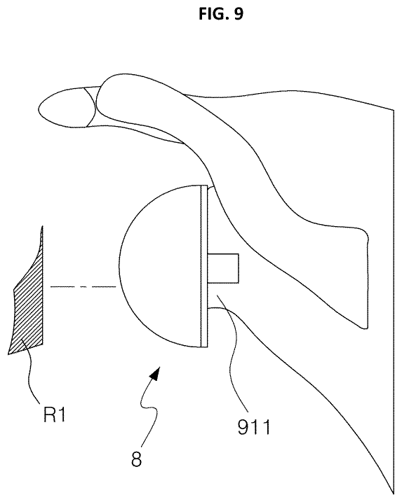

In another embodiment illustrated in FIG. 9, the reinforcement member may further include a second rib 135c. When the shaft 131 has the tapered shape, the second rib 135c extends from one end of the shaft 131 having a relatively large diameter to a predetermined portion, so that additional strength reinforcement is capable of being realized.

Referring to FIG. 3 to FIG. 6 and FIG. 11 again, the porous layer 30 is formed such that the porous layer 30 has the predetermined thickness from the base and a side of the insertion part and coats the surface of the base 11 and the surface of the insertion part 13, has a three-dimensional structure having a pores therein, and there is no specific limitation regarding the shape of the pores. The porous layer 30 may induce bone growth between pores inside the porous layer 30, so that union between a bone defect portion and bone fragments may be promoted or a postoperative strength that is difficult to realize with an artificial joint alone may be realized. The material constituting the porous layer 30 is not limited to any specific concept, but may preferably be titanium. The porous layer 30 may be formed by using titanium powder, alloy powder based on titanium, or the like and by using a 3D printing method. After the 3D printing and so on are performed, the porous layer 30 is manufactured by performing a post-process such as cleaning and so on. That is, the porous layer 30 forms pores on the outer surface of the upper surface 11a of the base and the insertion part 13 by using a biocompatible material powder such as titanium powder, titanium alloy powder, cobalt chromium powder, and so on, thereby being capable of increasing a coupling force with the bone using bone ingrowth into the pores when the porous layer 30 is implanted into the human body. The porous layer 30 has a complementary shape with the base and the insertion part, and includes a first layer 31 formed corresponding to the base 11 and a second layer 33 formed corresponding to the insertion part 13.

The first layer 31 is a portion formed on the surface of the base 11. Preferably, the first layer 31 is a portion formed on the upper surface 11a, the portion having a predetermined thickness. The first layer 31 includes a through-hole 311 and a protrusion portion 313. In another embodiment, the first layer 31 may be provided such that the first layer 31 is formed up to the side surface 11b and coats a circumference of the base 11.

The through-hole 311 is a portion in which an opening is formed to correspond to the peripheral fixing hole 113 that is formed so that the fixing means can pass therethrough. Referring to FIG. 6, in an exemplary embodiment, the through-hole 311 may be formed such that the through-hole 311 is larger than the peripheral fixing hole 113 by a predetermined amount, so that the flange 113a is capable of passing through the through-hole 311 and is capable of being exposed toward a direction toward the glenoid.

The protrusion portion 313 is formed corresponding to the recessed part 115 formed on a side of the upper surface 11a of the base described above, and may protrude by a height at which the recessed part 115 is recessed from the upper surface 11a such that the protrusion portion 313 has a complementary shape with the upper surface 11a. Accordingly, pores are increased, so that more bone growth is capable of being realized.

The second layer 33 is a portion formed by a predetermined thickness while surrounding the surface of the insertion part 13. That is, the second layer 33 is a portion surrounding the outer surface of the shaft 131 and the outer surface of the reinforcement member 135. Furthermore, the second layer 33 includes a recessed line 331 formed corresponding to the rib 135a and the rim 135b. It is preferable that the recessed line 331 is formed by being recessed by the protruding width and the protruding thickness of the rib 135a and the rim 135b.

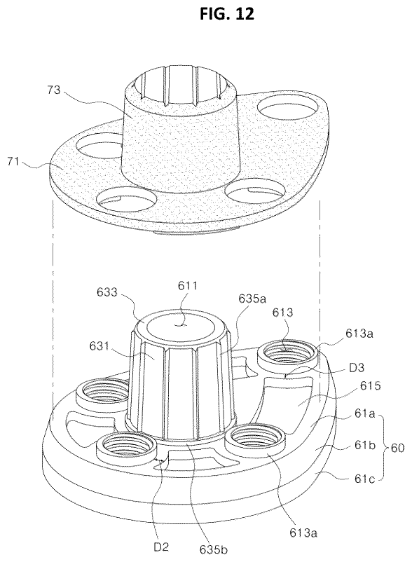

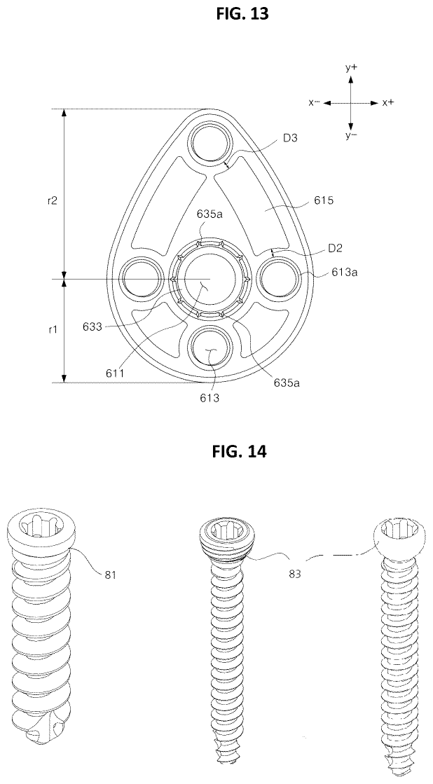

In describing a glenoid baseplate 6 according to another embodiment with reference to FIG. 12 and FIG. 13, the glenoid baseplate 6 has a non-circular shape or an asymmetric shape. Accordingly, the glenoid baseplate 6 may include a base 61 having a non-circular shape and/or an asymmetric shape, a solid region 60 having an insertion part 63, and a porous layer 70 coating the solid region 60 by a predetermined thickness corresponding to the solid region 60.

The base 61 may have an upper surface 61a, a side surface 61b, and a lower surface 61c. Furthermore, the base 61 may include a central fixing hole 611 formed in a center of the base 61, and may include a peripheral fixing hole 613 formed around the central fixing hole. Furthermore, a flange 613a may be formed on an edge of the peripheral fixing hole. In an embodiment, according to a shape of the glenoid, the base 61 may have a shape in which a side is elongated. Preferably, the base 61 may have a shape in which a superior side is elongated. Accordingly, a distance r1 from the center of the base 61 to one side may be different from a distance r2 from the center of the base 61 to the other side. At this time, the center of the base 61 may refer to a center of gravity of the base. In another embodiment, the center of the base 61 may refer to a center of the central fixing hole 611.

In more detailed description, referring to FIG. 12, a plane shape of the base 61 may be formed such that the base 61 extends in a y+ direction. Accordingly, a recessed part 615 formed by recessing a portion that is spaced apart from the peripheral fixing hole 613 and the central fixing hole 611 by a predetermined distance may be formed such that a shape of the recessed part 615 in a y+ side with respect to the central fixing hole 611 and a shape of the recessed part 615 in a y− side with respect to the central fixing hole 611 are different from each other. In the base 61 having the shape elongated in the y+ direction, a portion of the recessed part 615 disposed on the y+ side may be formed over a wider area than another portion of the recessed part 615 disposed on the y− side. Furthermore, as the stresses acting on each portion of the base 61 are different from each other, distances D2 and D3 that are the boundary of the recessed part may be different from each other. In an embodiment, in the boundary of the recessed part, the distance D2 spaced apart from the peripheral fixing hole 613 disposed in an x+ direction with respect to the central fixing hole 611 and a distance D3 spaced apart from the peripheral fixing hole 613 disposed in the y+ direction with respect to the central fixing hole 611 may be different from each other. The distance D2 spaced apart from the peripheral fixing hole 613 disposed in the x+ direction or x− direction may be smaller than the distance D3 spaced apart from the peripheral fixing hole 613 disposed in the y+ direction with respect to the central fixing hole 611. However, it is not excluded that the distance D2 spaced apart from the peripheral fixing hole 613 disposed in the x+ direction or x− direction is equal to or larger than the distance D3 spaced apart from the peripheral fixing hole 613 disposed in the y+ direction (D2>=D3).

In the embodiment, ribs 635a may be disposed at different center angles on the shaft 631. In FIG. 12, more ribs 635a may be disposed around a portion where stress is more distributed. Such arrangement of the ribs 635a may also be seen in the circular base 61. According to the movement of the shoulder joint, the ribs 635a may be disposed more densely in the y+ direction and/or the y− direction around the central fixing hole 611. That is, the ribs disposed in the y+ direction and/or the y− direction may be disposed at a center angle smaller than a center angle of the ribs disposed in the x+ direction and/or the x− direction.

Next, referring to FIG. 14, the fixing means 8 inserted into the glenoid baseplate 1 and configured to couple a scapular element and the scapula 91 will be described. The fixing means 8 may include a central fixing means 81 inserted into the central fixing hole 111, and may include an outer fixing means 83 inserted into the peripheral fixing hole 113. A thread is formed on an outer circumferential surface of the central fixing means 81, so that a coupling force with the scapula 91 may be sufficiently secured. In the outer fixing means 83, a thread is formed on a head portion of the outer fixing means 83, and the outer fixing means 83 is naturally induced to be coupled to the glenoid 911 in a predetermined direction. Preferably, the outer fixing means 83 is naturally induced to be coupled to the glenoid 911 in a direction perpendicular to the glenoid 911, so that an angle capable of providing the maximum coupling force may be rapidly and easily secured. In addition, since the head has a curved surface, the outer fixing means 83 may be inclined inward or outward from within the peripheral fixing hole 113, and may be inserted at various angles.

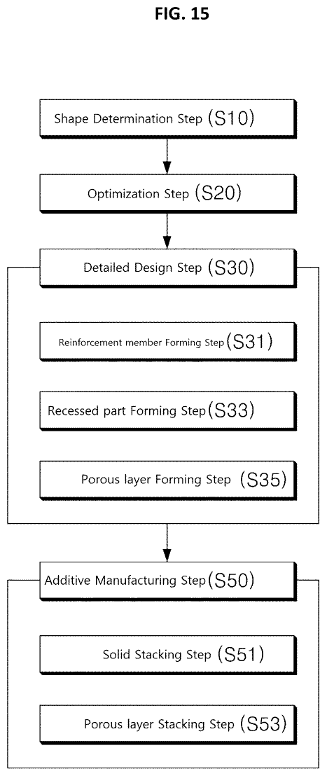

Hereinafter, a manufacturing method S of a glenoid baseplate according to an embodiment of the present disclosure will be described with reference to FIG. 15 to FIG. 18. In the manufacturing method S of the glenoid baseplate, a baseplate inserted into and/or mounted on a patient's glenoid is made lightweight while exhibiting a required strength, a portion that faces the bone is recessed and a porous layer is maximized, and the bone growth after the glenoid baseplate is mounted is maximized, so that a rapid recovery after surgery may be expected. The manufacturing method S of the glenoid baseplate includes a shape determination step S10, an optimization step S20, a detailed design step S30, and an additive manufacturing step S50.

The shape determination step S10 is a process of determining a shape of the glenoid baseplate, and is a process of determining a size and a shape, such as an overall shape of the glenoid baseplate 1, sizes, positions, and numbers of the central fixing hole 111 and the peripheral fixing hole 113, an extension length of the insertion part 13, and so on.

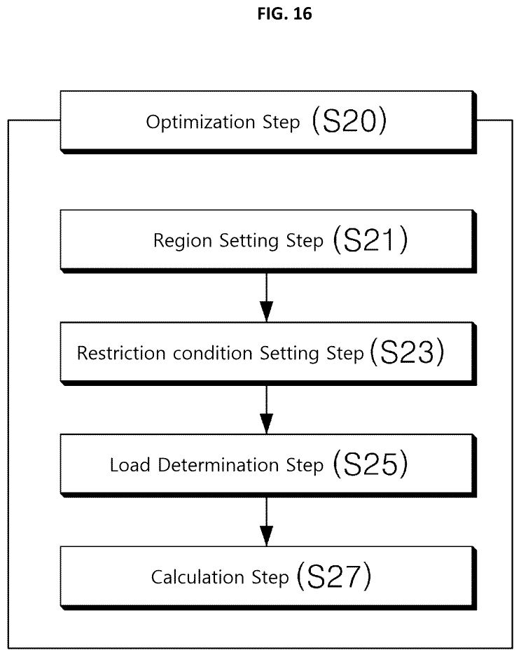

Referring to FIG. 16, the optimization step S20 is a process of deriving an optimal shape of the glenoid baseplate on the basis of a load and a restriction condition acting on the glenoid baseplate. In an embodiment, the optimal shape may be derived by using a phase optimization design method. The phase optimization design is a structural optimization design method in which connectivity of each element constituting a structure is optimized so that an objective function is capable of being realized while a design condition is satisfied. The phase optimization design may solve a problem in which a phase that occurs during the shape optimization process is fixed, and there is an advantage that a degree of freedom is increased. Therefore, in the manufacturing method S of the glenoid baseplate according to an embodiment of the present disclosure, the optimal shape of the baseplate may be derived on the basis of an input value and a constraint condition. At this time, in a state in which the position of the central fixing hole 111, the position of the peripheral fixing hole 113, the length of the shaft 131, and so on are determined in the shape determination step S10, the material used in the portion having less stress and less deformation is minimized so as to realize the optimal shape, so that the glenoid baseplate may be made lightweight and increased bone growth may be realized. The optimization step S20 may include a region setting step S21, a restriction condition setting step S23, a load determination step S25, and a calculation step S27. Furthermore, the optimization step S20 may be performed multiple times.

The region setting step S21 is a process of setting a region to be optimized through analysis. In the region setting step S21, a region to be optimized is designated so that the required strength is exhibited and the glenoid baseplate is made lightweight, so that a portion accommodating the fixing means or a portion in contact with the bone or the tissue may be excluded from the region to be optimized. In an exemplary embodiment of the present disclosure, portions of the central fixing hole 111, the peripheral fixing hole 113, and the flange 113a are set as an undesigned region, so that unnecessary degradation of the strength may be prevented. Particularly, in the region setting step S21, the glenoid baseplate 1 may be divided into several portions, and the optimization may be performed on one portion and then the optimization for other portions may be performed.

The restriction condition setting step S23 is a process of setting an optimization restriction condition together with an objective function that is an objective of the optimization, and the objective function may be set to exhibit the strength and the volume and the weight equal to or less than a predetermined degree may be set so that the glenoid baseplate is made lightweight. In an embodiment, the objective function of the glenoid base plate may be set to exhibit the strength capable of withstanding an applied load, and a volume equal to or less than 80% may be set as the restriction condition.

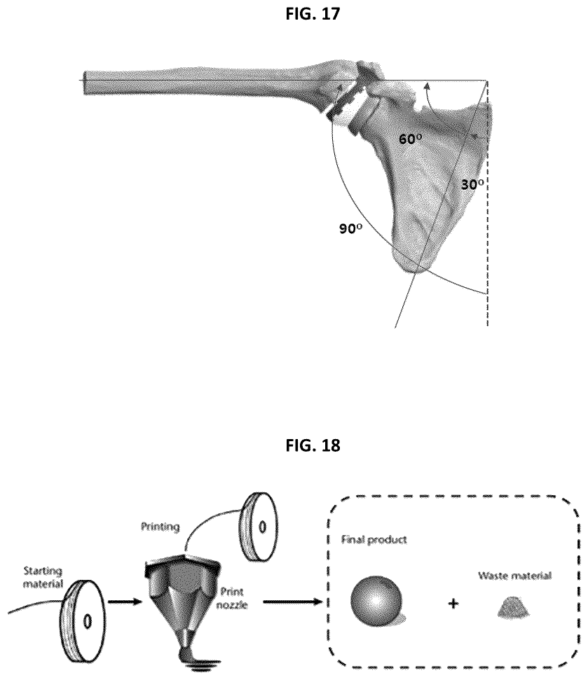

The load determination step S25 is a process of setting a load applied to the glenoid baseplate and a constraint condition. According to the movement of the shoulder joint, a load applied to the glenoid baseplate may be considered as multiple loads applied individually or applied in an overlapping manner. As a load, a concentrated load, a pressure, a forced displacement, and so on may be considered. As illustrated in FIG. 17, in an embodiment, the scapula is rotated approximately 30° in a 90° abduction, thereby forming a 60° scapulohumeral angle. Furthermore, in a state in which there is no supraspinatus, the maximum scapulohumeral joint force occurs near 90° abduction in an inverted prosthesis, so that a force may be applied at an angle of about 30° from the vertical with respect to the base. In addition, a compression force, a moment, and so on may occur according to a movement of the arm. At the same time, the constraint condition of the glenoid baseplate is set, and a predetermined region of the glenoid baseplate may be plane-constrained, hinge-constrained, and so on. In an embodiment of the present disclosure, a portion of the base may be plane-constrained and interpreted.

The calculation step S27 is a process of deriving the optimal shape through an analysis. Through this, the optimal shape of the glenoid baseplate that exhibits the proper strength while being made lightweight may be derived. As described above in the calculation step S27, the thickness or the width of the base 11 and the insertion part 13 may be optimized, and the recessed part 115 and the reinforcement member 135 may be formed. Particularly, the calculation step S27 may be substantially the same configuration as the detailed design step S30 that will be described later. In some embodiments, both the dimension and the cross-sectional shape of the recessed part 115 and the reinforcement member 135 may be determined in the calculation step S27, but may be additionally determined and considered in the detailed design step S30 for convenience in processing and additive manufacturing.

The detailed design step S30 is a process in which a detailed shape of the glenoid baseplate is determined according to the optimal shape determined in the optimization step. In an embodiment, the dimension and the cross-sectional shape of the recessed part 115 and the reinforcement member 135 may be determined and the thickness of the porous layer may be determined. The detailed design step S30 may include a reinforcement member forming step S31, a recessed part forming step S33, and a porous layer forming step S35.

The reinforcement member forming step S31 is a process of determining the reinforcement member formed around the insertion part of the glenoid baseplate. In the reinforcement member forming step S31, at least one of the cross-sectional shape, the number, the extension length, the center angle of the rib that protrudes and extends by the predetermined width from one end to the other end of the base of the shaft and the rim with an annular shape having the predetermined width at least the first end of the shaft and protruding from the shaft, thereby realizing the glenoid baseplate to be made lightweight while the rigidity of the portion of the shaft 131 is secured.

The recessed part forming step S33 is a process of determining the recessed part that is recessed from the base. In the recessed part forming step S33, at least one of the surface, the recessed depth, and the distance from the base may be determined.

The porous layer forming step S35 is a process of determining the porous layer that is formed so as to coat the surface of the solid region formed of the base and the insertion part. The porous layer 30 is formed complementary to the upper surface 11a of the base and the outer surface of the insertion part 13, thereby maximizing bone growth rate.

Referring to FIG. 18, the additive manufacturing step S50 is a process in which the determined solid region and the porous layer are manufactured by a stacking method. In the additive manufacturing step S50, metal powder may be supplied to an implant surface together with an auxiliary gas, and metal may be stacked by a thickness of several mm or more by performing melting with a laser heat source, thereby performing 3D printing. When the glenoid baseplate is formed in the additive manufacturing method, the glenoid baseplate may be stacking manufactured in an optimal size inducing the bone to grow well inside the structure, so that the bone ingrowth may be well realized, thereby being capable of increasing an initial fixation force. The additive manufacturing step S50 includes a solid stacking step S51 and a porous layer stacking step S53.

The solid stacking step S51 is a process of additive manufacturing the solid region 10 formed of the base 11 and the insertion part 13 determined through the process up to the detailed design step S30. The solid stacking step S51 is substantially a type of additive manufacturing, and may preferably be performed by using a 3D printer.

The porous layer stacking step S53 is a process of coating the solid region 10 with the porous layer. The porous layer stacked in the porous layer stacking step S53 has a shape complementary to one surface of the solid region 10. Furthermore, since the porous layer forms plural pores and is coated on the solid region, the density of the porous layer is lower than that of the solid region. Therefore, the baseplate is capable of being made lightweight, and bone growth may be increased since relatively more porosity is secured. In addition, in the porous layer stacking step S53, the porous layer and the solid region may be integrally stacked.

It is preferable that the solid region that is stacked in the solid stacking step S51 and the porous layer that is stacked in the porous layer stacking step S53 are stacked with the same material.

The foregoing detailed description is for illustrative purposes only. In addition, the description provides an exemplary embodiment of the present disclosure, and the present disclosure may be used in other various combination, changes, and environments. That is, the present disclosure may be changed or modified within the scope of the present disclosure described herein, a range equivalent to the description, and/or within the knowledge or technology in the related art. The embodiments show an optimum state for achieving the spirit of the present disclosure, and various modification required for specific applications and uses of the present disclosure are also possible. Therefore, the detailed description of the present disclosure is not intended to limit the present disclosure in the embodiment. In addition, the claims should be construed as including other embodiments.

1. A glenoid baseplate comprising:
a base having a predetermined thickness and being mounted on a glenoid of a scapula; and
an insertion part extending from one side of the base with a predetermined angle, wherein the insertion part comprises a shaft that extends from a surface of the base.

2. The glenoid baseplate of claim 1, wherein the insertion part comprises a reinforcement member that protrudes along an outer circumferential surface of the insertion part,
and the reinforcement member comprises a rib protruding and extending from one end of the base to the other end of the base of the shaft with a predetermined width.

3. The glenoid baseplate of claim 2, wherein the reinforcement member further comprises a rim protruding in a ring shape having a predetermined width from at least one end of the shaft.

4. The glenoid baseplate of claim 3, wherein the width of the rib is gradually decreased as the rib protrudes from an outer surface of the shaft.

5. The glenoid baseplate of claim 1, comprising a recessed part recessed by a predetermined depth in at least one surface of the base.

6. The glenoid baseplate of claim 5, wherein the base comprises a central fixing hole formed vertically through the base, and a peripheral fixing hole formed around the central fixing hole, and
the recessed part is recessed on a surface of the base at least a predetermined distance from the edge of at least one of the central fixing hole, the peripheral fixing hole, and the edge of the base.

7. The glenoid baseplate of claim 6, wherein the base further comprises a flange protruding from one surface of the base along the edge of the peripheral fixing hole.

8. The glenoid baseplate of claim 1, comprising a porous layer being formed such that the porous layer coats surfaces of the base and the insertion part with a predetermined thickness and having plural pores therein,
wherein the porous layer has a complementary shape to the base and the insertion part.

9. The glenoid baseplate of claim 8, wherein an extended end of the shaft and the edge of the fixing hole vertically passing through the base are exposed without being covered by the porous layer.

10. A manufacturing method of a glenoid baseplate, the manufacturing method comprising:
a shape determination step determining a shape of the glenoid baseplate;
an optimization step deriving an optimal shape of the glenoid baseplate on the basis of a load and a restriction condition acting on the glenoid baseplate;
a detailed design step determining a detailed shape of the glenoid baseplate according to the optimal shape determined in the optimization step; and
a additive manufacturing step manufacturing a determined solid region and a determined porous layer in a stacking manner.

11. The manufacturing method of claim 10, wherein the optimization step comprises:
a region setting step setting a region to be optimized through analysis;
a restriction condition setting step setting an optimization restriction condition together with an objective function that is an objective of optimization;
a load determination step setting a load and a constraint condition applied to the glenoid; and
a calculation step deriving the optimal shape.

12. The manufacturing method of claim 11, wherein the detailed design step comprises:
a reinforcement member forming step determining a reinforcement member formed around an insertion part of the glenoid baseplate;
a recessed part forming step determining a recessed part formed by being recessed from a base of the glenoid baseplate; and
a porous layer forming step determining a porosity and a thickness of the porous layer that is formed so as to coat a surface of the solid region formed of the base and the insertion part.

13. The manufacturing method of claim 12, wherein the detailed design step includes a reinforcement member forming step determining a reinforcement member formed around the insertion part of the glenoid baseplate, a recessed part forming step determining a recessed part forming from the base of the glenoid baseplate, and a porous layer forming step determining the pores and thickness of a porous layer formed to coat the surface of a solid region including the base and the insertion part.

14. The manufacturing method of claim 12, wherein, the recessed part forming step determines at least one of a surface, a recessed depth, and a distance from the base.

15. The manufacturing method of claim 10, wherein the solid region and the porous layer are stacked with the same material.
